(19) United States
(12) Patent Application Publication
(10) Pub. No.: US 2025/0049409 A1
(43) Pub. Date: Feb. 13, 2025

(54) METHODS OF QUANTIFYING SKELETAL MUSCLE PERFUSION USING PET IMAGING

(71) Applicant: Research Institute at Nationwide Children’s Hospital, Columbus, OH (US)

(72) Inventors: Mitchel Stacy, Columbus, CO (US); Ting-Heng Chou, Columbus, OH (US)

(21) Appl. No.: 18/800,902

(22) Filed: Aug. 12, 2024

Related U.S. Application Data
(60) Provisional application No. 63/519,098, filed on Aug. 11, 2023.

(51) Int. Cl. A61B 6/50; A61B 6/03; A61K 51/02; A61K 51/04

(52) U.S. Cl. CPC A61B 6/507; A61B 6/037; A61K 51/025; A61K 51/0491

(57) ABSTRACT

Described herein are methods for quantifying skeletal muscle perfusion, skeletal muscle metabolism, and active vascular calcification by positron emission tomography (PET) imaging of labeled radionuclides, such as ¹⁸F-labeled radionuclide agents.

### CROSS-REFERENCE TO RELATED APPLICATIONS

This application claims benefit of and priority to U.S. Ser. No. 63/519,098 filed Aug. 11, 2023, which is incorporated by reference in its entirety.

### U.S. GOVERNMENT SUPPORT

This invention was made with government support under HL135103 awarded by the National Institutes of Health. The government has certain rights in the invention.

### FIELD OF THE INVENTION

This invention is generally in the field of quantifying skeletal muscle perfusion, skeletal muscle metabolism, and active vascular calcification by imaging of labeled radionuclides.

### BACKGROUND OF THE INVENTION

Lower extremity peripheral artery disease (PAD) is an atherosclerotic disease affecting more than 230 million people globally that significantly impairs lower extremity function and quality of life through reductions in skeletal muscle blood flow and perfusion (Misra S, et al., Circulation. 2019; 140:e657-e672; Criqui M H, et al., Circulation. 2021; 144:e171-e191). These impairments to muscle perfusion subsequently contribute to an increased risk of amputation as well as increased rates of morbidity and mortality for PAD patients (Criqui M H, et al., Circulation. 2021; 144:e171-e191). Despite the significant role of perfusion in PAD, the vascular medicine community lacks a standard method for quantifying absolute measures of muscle perfusion in PAD patients.

Recent imaging studies using nuclear imaging-based approaches have demonstrated that single-photon emission computed tomography/computed tomography (SPECT/CT) imaging can detect abnormalities in resting muscle perfusion in PAD patients (Alvelo J L, et al., Circ Cardiovasc Imaging. 2018; 11:e006932) and quantify improvements in relative perfusion following endovascular revascularization (Chou T H, et al., Adv Wound Care. 2020; 9:103-110) that may predict risk of amputation (Chou T H, et al., JACC Cardiovasc Imaging. 2021; 14:1614-1624). Although SPECT/CT has demonstrated utility for assessing relative perfusion in PAD, SPECT imaging still suffers from various limitations, such as limited spatial and temporal resolution, lack of equipment and image analysis tools for quantifying absolute measures of perfusion, and generally higher radiation exposure compared to positron emission tomography (PET) imaging.

Compared to SPECT, PET offers improved spatial and temporal resolution, higher sensitivity count detection, and generally less radiation exposure with the use of shorter half-life radioisotopes. (Burchert W, et al., J Nucl Med. 1997; 38:93-98; Schmidt M A, et al., J Nucl Med. 2003; 44:915-919; Scremin O U, et al., Am J Phys Med Rehabil. 2010; 89:473-486; Fischman A J, et al., J Appl Physiol. 2002; 92:1709-171). However, traditional PET perfusion radioisotopes, such as rubidium-82, oxygen-15 water (¹⁵O-water), and nitrogen-13-ammonia possess short half-lives (1.273 min, 2.04 min, and 9.8 min, respectively) which necessitates on-site generator or cyclotron production and rapid dose injections by well-trained radiochemistry teams.

Thus, there remains a need in the vascular medicine community for new and noninvasive methods for quantifying absolute measures of muscle perfusion (e.g., mL/min/100 g) in subjects which can overcome known issues with currently used methods.

Thus, it is an object of the present invention to provide methods for quantifying absolute measures of skeletal muscle perfusion in a subject.

It is a further object of the present invention to provide methods for quantifying skeletal muscle metabolism in a subject.

It is yet another object of the present invention to provide methods for quantifying active vascular calcification in a subject.

### SUMMARY OF THE INVENTION

Methods of quantifying muscle perfusion in a subject using labeled radionuclides, as well as methods of quantifying active vascular calcification in a subject are described herein. In one non-limiting instance, such a method includes the steps of:
(i) administering to a subject an effective amount of a fluorine-18-labeled radionuclide agent;
(ii) dynamically imaging the subject on a positron emission tomography (PET) system, wherein the imaging begins at least about 1 second prior to step (i); and
(iii) performing kinetic modeling of the PET imaging data following steps (i) and (ii).

Dynamic PET imaging in step (ii) involves the continuous imaging over a pre-defined period immediately after administration. Tracer kinetic modeling based on several different models allows for parametric mapping as well as the extraction of time-activity curves (TACs) for arteries and the selected volumes of interest.

In some instances, the kinetic modeling of step (iii) is one-tissue compartment or three-tissue compartment kinetic modeling.

In some instances, the skeletal tissue is a muscle or group of muscles of the subject. In some instances, the muscle or group of muscles is selected, without particular limitation, from extremity, back, gluteus, abdominal, neck, and foot muscles.

In some instances, the above method further includes, subsequent to step (ii):

(iv) generating at least one pixelwise perfusion map of the tissue, by parametric mapping.

In still other instances, the method can further include, subsequent to step (iv):

(v) evaluating the at least one pixelwise perfusion map of the tissue.

The method described can quantify skeletal muscle perfusion in a tissue of a subject.

In some instances, the subject suffers from a disease including but not limited to, a neuromuscular disease, an ischemic disease, and/or a degenerative muscle disease. In some instances, the subject suffers from one or more diseases which may include, without limitation, peripheral artery disease (PAD), Charcot-Marie-Tooth syndrome (CMT), exertional compartment syndrome, ischemic myopathy, ischemic rhabdomyolysis, chronic limb-threatening ischemia (CLTI), ischemic necrosis, ischemic fasciitis, ischemic myositis, and combinations thereof.

In some instances, the method described above may be modified and used to quantify skeletal muscle metabolism in a subject, such as when the fluorine-18-labeled radionuclide agent is fluorine-18-labeled glucose or analog thereof. In such instances, the imaging of step (ii) is carried out for a longer period of time, such as for about 5 to 60 minutes. In some instances, a method for quantifying skeletal muscle metabolism in a subject can include the steps of:
(i′) administering to the subject an effective amount of fluorine-18-labeled glucose or analog thereof; and
(ii′) dynamically imaging the patient on a positron emission tomography (PET) system for about 5 to 60 minutes following step (i′); and
(iii′) performing three-tissue compartment kinetic modeling of the PET imaging data following steps (i′) and (ii′).

Also disclosed herein are methods for quantifying active vascular calcification using labeled radionuclide agents, such as when the fluorine-18-labeled radionuclide agent is fluorine-18-labeled sodium fluoride. In one non-limiting instance, the method includes the steps of:
(a) administering to a subject an effective amount of a fluorine-18-labeled sodium fluoride;
(b) dynamically imaging the subject using a positron emission tomography (PET) system; and
(c) following step (b), imaging the subject a second time using the PET system, wherein a second dose of a fluorine-18-labeled sodium fluoride or another fluorine-18-labeled radionuclide agent is not administered to the subject between step (b) and (c). In step (c) the second PET imaging step typically involves acquiring image data over a single time frame (static imaging). In some instances, a method for quantifying active vascular calcification on a subject can be carried out in combination with a method for quantifying skeletal muscle perfusion in a subject.

In some instances, the vascular calcification is quantified in a blood vessel, such as a vein or artery, of the subject, such as for example the aorta, inferior vena cava, carotid artery, carotid vein, coronary artery, brachial artery, brachial vein, radial artery, radial vein, femoral artery, femoral vein, popliteal artery, popliteal vein, anterior tibial artery, posterior tibial artery, tibioperoneal trunk, peroneal artery, dorsalis pedis artery, renal artery, or renal vein.

### BRIEF DESCRIPTION OF THE DRAWINGS

Non-limiting embodiments are described by way of example with reference to the accompanying Figures.

FIG. 1 is a bar graph of the quantitative ¹⁸F-NaF PET/CT image analysis of calf muscle perfusion (mL/min/100 g) in porcine model of PAD which confirmed a significant decrease in resting calf muscle perfusion in the occluded hindlimb (gray bar) that normalized to control (black bar) levels two weeks after arterial occlusion. Serial changes in perfusion were evaluated using a two-way repeated measures analysis of variance (ANOVA). N=8 pigs.

FIG. 2A is a bar graph of the capillary density (capillary to fiber ratio) for both the gastrocnemius and soleus muscles of the ischemic hindlimb comparing control limb (darker bar) to ischemic limb (lighter bar) 2 weeks after arterial occlusion, indicating that compensatory microvascular remodeling in the setting of hindlimb ischemia aligned with quantitative improvements in muscle perfusion. Data are shown as mean±SEM, circles represent individual values for each control limb and squares represent individual values for ischemic limb. Differences in capillary density between control and ischemic muscles were evaluated using paired t-tests. N=4 pigs.

FIG. 2B is a bar graph of the arteriole density (% positive α-SMA area) for both the gastrocnemius and soleus muscles of the ischemic hindlimb comparing control calf (darker bar) to ischemic calf (lighter bar) 2 weeks after arterial occlusion, indicating that compensatory microvascular remodeling in the setting of hindlimb ischemia aligned with quantitative improvements in muscle perfusion. Data are shown as mean±SEM, circles represent individual values for each control limb and squares represent individual values for ischemic limb. Differences in arteriole density between control and ischemic muscles were evaluated using paired t-tests. N=4 pigs. α-SMA=alpha-smooth muscle actin.

FIG. 3A is a graph of the measurements of muscle perfusion (mL/min/100 g) obtained in a porcine model of PAD using microsphere-based assessment versus dynamic ¹⁸F-NaF PET perfusion (mL/min/100 g), showing that the PET-derived perfusion measurements agreed with microsphere-derived perfusion measurements, with a concordance correlation coefficient (CCC) of 0.85 (95% CI: 0.76, 0.90) between perfusion measures. N=4 pigs.

FIG. 3B is a graph of the Bland-Altman analysis which revealed a mean difference of 0.52 with 95% agreement limits of −1.19 to 2.2 for mean muscle perfusion measurements (mL/min/100 g) acquired by dynamic ¹⁸F-NaF PET and the gold standard microsphere method. N=4 pigs.

FIGS. 4A, 4B, and 4C are bar graphs of ¹⁸F-NaF PET-derived skeletal muscle perfusion values (mL/min/100 g) for different muscles in the calf muscle (gastrocnemius, soleus, and tibialis anterior) after grouping of patients based on claudication symptoms and disease stage (i.e., Rutherford Classification). Data are shown as mean±SEM (individual values for each patient are represented with circles). Stage 0-1, N=15 patients; Stage 2-3, N=12 patients; Stage 4-6, N=12 patients. Differences in calf perfusion for each Rutherford classification category were compared using one-way ANOVA, with Tukey's method used to account for multiple comparisons. FIG. 4A is a bar graph of the PET-derived skeletal muscle perfusion values (mL/min/100 g) for the gastrocnemius calf muscle in the patient groups. FIG. 4B is a bar graph of the PET-derived skeletal muscle perfusion values (mL/min/100 g) for the soleus calf muscle in the patient groups. FIG. 4C is a bar graph of the PET-derived skeletal muscle perfusion values (mL/min/100 g) for the tibialis anterior calf muscle in the patient groups.

FIG. 5 is a graph of a quantitative ¹⁸F-NaF PET/CT image analysis demonstrating regional improvements in perfusion (mL/min/100 g) after lower extremity intervention for individual muscles of the calf (gastrocnemius, soleus, and tibialis anterior, circles representing pre-intervention and squares representing post-intervention perfusion values).

FIG. 6 is a bar graph of quantitative ¹⁸F-FDG PET/CT image analysis of perfusion within specific muscles of the calf (gastrocnemius, soleus, and tibialis anterior) which demonstrated significantly reduced skeletal muscle perfusion (mL/min/100 g) for multiple muscle groups of the lower extremity in patients with Charcot-Marie-Tooth (CMT) disease. Data are shown as mean±SEM. N=8 healthy control patients (dark grey bar) and N=4 CMT patients (light grey bar), circles representing individual values for each CMT patient and squares representing individual values for each healthy control patient.

FIG. 7 is a bar graph of the serial evaluation of calf muscle metabolism in a porcine model of hindlimb ischemia using dynamic ¹⁸F-FDG PET/CT imaging. Quantitative PET/CT image analysis demonstrated that the metabolic rate of glucose was significantly reduced in the calf muscle of the ischemic limb on the day of arterial occlusion and recovered to control levels 2 weeks later. N=7 pigs.

### DETAILED DESCRIPTION OF THE INVENTION

Methods of quantifying muscle perfusion in a subject using labeled radionuclides are described herein. Further described are methods of quantifying active vascular calcification in a subject.

### I. Definitions

“Fluorine-18 labeled radionuclide agent,” as used herein, refers to a pharmaceutically acceptable compound having a fluorine-18 (¹⁸F) label attached thereto or associated therewith.

The term “subject” typically refers to a mammal, such as an animal or human.

The term “effective amount” as used herein with respect to the amount of a radionuclide agent generally refers to the amount of a radionuclide that is sufficient for purposes of performing imaging, such as with a positron emission tomography (PET) system, in a subject to whom the radionuclide was administered. The actual effective amounts of a radionuclide agent can vary according to the specific radionuclide(s) being utilized, the particular composition formulated for administration, the mode of administration, and/or the age, weight, and condition of the subject.

Numerical ranges disclosed in the present application include, but are not limited to, ranges of temperatures, ranges of pressures, ranges of molecular weights, ranges of integers, ranges of force values, ranges of times, ranges of thicknesses, and ranges of gas flow rates. The disclosed ranges of any type, disclose individually each possible number that such a range could reasonably encompass, as well as any sub-ranges and combinations of sub-ranges encompassed therein. For example, disclosure of a temperature range, is intended to disclose individually every possible temperature value that such a range could encompass, consistent with the disclosure herein. In another example, the disclosure states that conformality, expressed as a percentage, can range from about 50% to about 90%, which also refers to percentage values that can be selected independently from about 57%, 68%, and 79%, as well as any sub-range between these numbers (for example, about 65% to 85%), and any possible combination of ranges between these values.

### II. Methods of Quantifying Muscle Perfusion

The methods described herein are able to overcome many of the economic and logistic issues associated with the use of short half-life radionuclides that are produced by onsite cyclotrons or generators. The methods described herein utilize a radionuclide agent that possesses a relatively long half-life, such as a fluorine-18 (¹⁸F)-labeled radionuclide, with a half-life of 109.7 minutes. Thus, the disclosed methods allow for unit dose ordering and shipment from nearby commercial vendors, and/or permit the use of PET perfusion imaging at a location within several hours of a commercial vendor of radiopharmaceuticals.

Disclosed herein are methods for quantifying skeletal muscle perfusion using labeled radionuclides.

### A. Methods for Quantifying Skeletal Muscle Perfusion

In one non-limiting instance, a method includes the steps of:
(i) administering to a subject an effective amount of a fluorine-18-labeled radionuclide agent;
(ii) dynamically imaging the subject on a positron emission tomography (PET) system, wherein the imaging begins at least about 1 second prior to step (i); and
(iii) performing kinetic modeling of the PET imaging data following steps (i) and (ii).

In some instances, the positron emission tomography (PET) system is a PET/computed tomography (PET/CT) or a PET/magnetic resonance (PET/MR) system.

In some instances, the kinetic modeling of step (iii) is one-tissue compartment or three-tissue compartment kinetic modeling.

In some instances, the fluorine-18 labeled radionuclide agent is ¹⁸F-sodium fluoride (NaF). NaF is an established, commercially available PET radionuclide for quantifying bone perfusion and metabolism (Czernin J, et al., J Nucl Med. 2010; 51:1826-1829; Piert M, et al., Eur J Nucl Med Mol Imaging. 2002; 29:907-914.) and active vascular microcalcification (Hoilund-Carlsen P F, et al., Eur J Nucl Med Mol Imaging. 2020; 47:1538-1551; Stacy M R. Front Med. 2022; 8:793975) due to its high affinity for hydroxyapatite.

In some instances, the fluorine-18 labeled radionuclide agent is fluorine-18-labeled glucose or a glucose analog, such as ¹⁸F-2-deoxy-2-fluoro-D-glucose (FDG). Other fluorine-18-labeled glucose analogs include, without limitation, ¹⁸F-FDGal (fluorodeoxygalactose), ¹⁸F-FDMan (fluorodeoxymannose), ¹⁸F-FET (fluoroethyltyrosine), and ¹⁸F-FDOPA (fluorodihydroxyphenylalanine). In particular, FDG is a glucose analog that tends to accumulate in a tissue, or group of cells, with high metabolic demand, such as tumors and inflammatory cells. FDG is a standard radiotracer commonly used for PET neuroimaging and diagnosis/monitoring of cancer patients.

In some other instances, the fluorine-18 labeled radionuclide agent can be selected from, without limitation, fluorocholine, flutemetamol, fluorothymidine, florbetapir, fluoropropyl-dihydrotetrabenazine, fluoromisonizadole, fluorobenzyl triphenyl phosphonium, fluoroethyl tyrosine, fluoropropyl-donepezil, 18-F-DCFPYL (2-(3-{1-carboxy-5-[(6-[18F]fluoro-pyridine-3-carbonyl)-amino]-pentyl}-ureido)-pentanedioic acid), or a combination thereof.

In some other instances, the various methods described herein may substitute a fluorine-18-labeled radionuclide agent with another suitable radionuclide agent. For instance, a gallium-68 labeled radionuclide agent can be used. Gallium-68 has a half-life of approximately one hour (about 68 minutes) and can be purchased and shipped from commercial vendors of radiopharmaceuticals. Without limitation, some examples of gallium-68 agents that could be used in the methods described herein include gallium-68 dotatate (Ga-68 DOTATATE), gallium-68 dotatoc (Ga-68 DOTATOC), gallium-68 PSMA-11 (Ga-68 PSMA-11), gallium-68 ProstateScan (Ga-68 ProstaScint), gallium-68 Pentixafor (Ga-68 Pentixafor), gallium-68 FAPI (fibroblast activation protein inhibitor), gallium-68 PSMA-617 (Ga-68 PSMA-617), gallium-68 Folate (Ga-68 Folate), gallium-68 NODAGA-RGD (Ga-68 NODAGA-RGD), gallium-68 Pentixather (Ga-68 Pentixather), gallium-68 Depreotide (Ga-68 Depreotide), gallium-68 Sestamibi (Ga-68 Sestamibi), gallium-68 Citrate (Ga-68 Citrate), gallium-68 Etarfolatide (Ga-68 Etarfolatide), gallium-68 RM2 (Ga-68 RM2), and gallium-68 Pentixafor (Ga-68 Pentixafor).

In some instances, dynamic imaging of the subject is performed for a period of time ranging from at least about 2 to about 10 minutes, or any sub-ranges within this time period. In some cases, the imaging is performed for at least about 2, 3, 4, 5, 6, 7, 8, 9, or 10 minutes. In some instance, the imaging begins at least about 1 to about 5 seconds prior to step (i), or any sub-ranges within this time period. In some cases, the imaging begins at least about 2, 3, 4, or 5 seconds prior to step (i).

Dynamic PET imaging in step (ii) involves the continuous imaging over a pre-defined period immediately after administration, such as from at least about 2 to about 10 minutes, thereby acquiring kinetic information. Tracer kinetic modelling based on several different models allows for parametric mapping as well as the extraction of time-activity curves (TACs) for arteries and the selected volumes of interest.

In some instances, during step (i) an effective amount of the fluorine-18 labeled radionuclide agent is administered to provide a dose in a range of about 1 mCi to about 10 mCi, or any sub-ranges within this dosage range, during at least the first 20 seconds of step (i), or any sub-ranges or individual time periods within this time period. In some instances, during step (i) an effective amount of the fluorine-18 labeled radionuclide agent is administered to provide a dosage of at least about 1 mCi, 2 mCi, 3 mCi, 4 mCi, 5 mCi, 6 mCi, 7 mCi, 8 mCi, 9 mCi, or 10 mCi, during at least the first 20 seconds of step (i), or any sub-ranges or individual time periods within this time period.

In some instances, the skeletal tissue is a muscle or group of muscles of the subject. In some instances, the muscle or group of muscles is selected, without particular limitation, from extremity, back, gluteus, abdominal, neck, and foot muscles.

Administering one or more types of fluorine-18-labeled radionuclide agents to a subject during step (i) can be performed by any suitable means. In some instances, step (i) is performed by administering the fluorine-18-labeled radionuclide agent via intra-venous or intra-arterial injection or infusion. Methods and conditions needed for administering agents, such as fluorine-18-labeled radionuclide agents, in suitable pharmaceutical formulation and at desired dosages are known the person of ordinary skill in the art.

In some instances, the above method further includes, subsequent to step (ii):
(iv) generating at least one pixelwise perfusion map of the tissue, by parametric mapping.

In still other instances, the method can further include, subsequent to step (iv):
(v) evaluating the at least one pixelwise perfusion map of the tissue.

The method described herein quantifies skeletal muscle perfusion in a tissue of a subject. Skeletal muscle perfusion is typically expressed in units of ml/min/g (or ml/min/100 g).

In some instances, the subject suffers from a disease including but not limited to, a neuromuscular disease, an ischemic disease, and/or a degenerative muscle disease. In some instances, the subject suffers from one or more diseases which may include, without limitation, peripheral artery disease (PAD), Charcot-Marie-Tooth syndrome (CMT), exertional compartment syndrome, ischemic myopathy, ischemic rhabdomyolysis, chronic limb-threatening ischemia (CLTI), ischemic necrosis, ischemic fasciitis, ischemic myositis, and combinations thereof. The methods described herein represent a translatable and scalable imaging approach capable of quantifying regional muscle perfusion in a subject, such as a PAD patient. The methods described herein can be used in a subject to improve screening and diagnosis of a neuromuscular disease, an ischemic disease, and/or a degenerative muscle disease, assisting in clinical decision making, and assessing perfusion responses to a therapy, such as emerging limb-saving therapies.

In some instances, a subject may be monitored prior to and following one or more medical procedures or therapies, where the method described herein is repeated during suitable time intervals, such as about 1 day to about 1 year or any subrange or individual value within this time interval, following the one or more medical procedures or therapies. In certain instances, the above method is performed on a subject prior to and following a surgical or endovascular revascularization procedure. In some instances, the method is repeated about 1 to about 30 days or any subrange or individual value within this time interval following the surgical or endovascular revascularization procedure. In some instances, the surgical or endovascular revascularization procedure is or involves a balloon angioplasty or deployment of a stent in the subject. In certain other instances, the above method is performed on a subject prior to and following a regenerative medicine therapy. In some instances, the method is repeated about 1 day to about 1 year or any subrange or individual value within this time interval following the surgical or endovascular revascularization procedure. In some instances, the regenerative medicine therapy is or involves a stem cell or gene therapy given to the subject. Other forms of regenerative medicine therapy may also be administered to the subject.

### B. Methods for Quantifying Skeletal Muscle Metabolism

In some instances, the methods described above in Section A may be modified and used to quantify skeletal muscle metabolism in a subject, such as when the fluorine-18-labeled radionuclide agent is fluorine-18-labeled glucose or analog thereof. In such instances, the imaging of step (ii) is carried out for a longer period of time, such as for about 5 to 60 minutes. In some instances, a method for quantifying skeletal muscle metabolism in a subject can include the steps of:
(i′) administering to the subject an effective amount of fluorine-18-labeled glucose or analog thereof; and
(ii′) dynamically imaging the patient on a positron emission tomography (PET) system for about 5 to 60 minutes following step (i′); and
(iii′) performing three-tissue compartment kinetic modeling of the PET imaging data following steps (i′) and (ii′).
   Imaging step (ii′) can be carried out for about 5 to 60 minutes, or any sub-range or individual time values within this range.

Details of the administration and PET system are provided above in Section A. In some instances, the method may also include kinetic modeling, such as one-tissue compartment kinetic modeling, of the PET imaging data following steps (i′) and (ii′), and this modeling step may be carried before or after step (iii′) and following steps (i′) and (ii′). Accordingly, in certain instances, a single method may be used for quantifying skeletal muscle perfusion and for quantifying skeletal muscle metabolism using a single administration of a fluorine-18-labeled glucose or analog thereof, such as ¹⁸F-2-deoxy-2-fluoro-D-glucose (FDG).

In some instances, the fluorine-18-labeled glucose agent or analog thereof is ¹⁸F-2-deoxy-2-fluoro-D-glucose (FDG), ¹⁸F-FDGal (fluorodeoxygalactose), ¹⁸F-FDMan (fluorodeoxymannose), ¹⁸F-FET (fluoroethyltyrosine), or ¹⁸F-FDOPA (fluorodihydroxyphenylalanine). In some instances, the fluorine-18-labeled glucose agent or analog thereof is ¹⁸F-2-deoxy-2-fluoro-D-glucose (FDG).

In some instances, during step (i′) an effective amount of the fluorine-18-labeled glucose or analog thereof is administered to provide a dose in a range of about 1 mCi to about 10 mCi, or any sub-ranges within this dosage range. In some instances, during step (i′) an effective amount of the fluorine-18-labeled glucose or analog thereof is administered to provide a dosage of at least about 1 mCi, 2 mCi, 3 mCi, 4 mCi, 5 mCi, 6 mCi, 7 mCi, 8 mCi, 9 mCi, or 10 mCi, during at least the first 20 seconds of step (i′), or any sub-ranges or individual time periods within this time period.

### C. Kinetic Modeling

Suitable software and methods for performing and evaluating compartment kinetic modeling of PET imaging data, such as the data generated during the methods described above in Sections A and B, are known to those skilled in the art. For example, one-tissue compartment kinetic modeling in PET is described in detail by Richard E. Carson in Tracer Kinetic Modeling in PET in Positron Emission Tomography: Basic Science and Clinical Practice. Springer-Verlag London Ltd 2003 by Valk, P E, et al. Three-tissue compartment kinetic modeling in PET, for instance, is described in detail in Kinetic modeling of [¹⁸F]FDG in skeletal muscle by PET in Am. J. Physiol. Endocrinol. Metab. 281: E524-E536, 2001 by A. Bertoldo, et al.

Optionally, in step (iii) and/or step (iii′), one-, two-, or three-compartment modeling is performed, as needed. Optionally, multi-compartment modeling using more than three volumes of interest (VOI), such as four-compartment modeling, within a tissue can be performed.

Other types of kinetic modeling of PET imaging data can also be used, in lieu of the one-, two-, or three-compartment modeling discussed above. Without limitation, additional kinetic modeling techniques include: Linear Models (Logan Plot, Patlak Plot); Nonlinear Models (Nonlinear Least Squares (NLLS), Bayesian Methods); Simplified Models (Standardized Uptake Value (SUV), Simplified Reference Tissue Model (SRTM)); Spectral Analysis (Basis Function Method (BFM)); and Data-Driven Models (Factor Analysis of Dynamic Structures (FADS), Graphical Analysis).

### III. Methods of Quantifying Active Vascular Calcification

Also disclosed herein are methods for quantifying active vascular calcifications using labeled radionuclide agents, such as when the fluorine-18-labeled radionuclide agent is fluorine-18-labeled sodium fluoride. In one non-limiting instance, the method includes the steps of:
(a) administering to a subject an effective amount of a fluorine-18-labeled sodium fluoride;
(b) dynamically imaging the subject using a positron emission tomography (PET) system; and
(c) following step (b), imaging the subject a second time using the PET system, wherein a second dose of a fluorine-18-labeled sodium fluoride or another fluorine-18-labeled radionuclide agent is not administered to the subject between step (b) and (c). In step (c) the second PET imaging step typically involves acquiring image data over a single time frame (static imaging). In some instances, a method for quantifying active vascular calcification on a subject can be carried out in combination with a method for quantifying skeletal muscle perfusion in a subject, as discussed in Section II above.

In some instances, the positron emission tomography (PET) system is a PET/computed tomography (PET/CT) or a PET/magnetic resonance (PET/MR) system.

In some instances, dynamic imaging of the subject on the PET system in step (b) is performed at least about 10 seconds to about 20 minutes following step (a), as well as sub-ranges disclosed within. In some instance, the imaging of step (b) can begin at least about 1 to about 5 seconds prior to step (a), as well as sub-ranges disclosed within. In some cases, the imaging of step (b) begins at least about 2, 3, 4, or 5 seconds prior to step (a). The dynamic imaging of step (b) can be performed for any suitable amount of time. In some cases, the imaging of step (b) is performed for at least about 20 seconds, 30 seconds, 40 seconds, 50 seconds, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 minutes.

In some instances, the second imaging step of step (c) is performed at least about 30 minutes to 75 minutes or 45 minutes to 75 minutes following the administration of the fluorine-18-labeled sodium fluoride in step (a). The second imaging of step (c) can be performed for any suitable amount of time, such as for about 10 seconds to about 30 minutes, or any sub-ranges disclosed within. In some cases, the second imaging of step (c) is performed for at least about 20 seconds, 30 seconds, 40 seconds, 50 seconds, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 minutes. The imaging step in step (c) can involve acquiring image data over a single time frame (static imaging).

In some instances, during step (a) the effective amount administered of the fluorine-18-labeled sodium fluoride provides a dose in a range of about 1 mCi to about 10 mCi, as well as sub-ranges disclosed within, during at least the first 20 seconds of step (a), as well as sub-ranges or individual time values disclosed within. In some instances, during step (i) the effective amount administered of the fluorine-18-labeled sodium fluoride provides a dosage of at least about 1 mCi, 2 mCi, 3 mCi, 4 mCi, 5 mCi, 6 mCi, 7 mCi, 8 mCi, 9 mCi, or 10 mCi, during at least the first 20 seconds of step (a), as well as sub-ranges or individual time values disclosed within.

In some instances, the vascular calcification is quantified in a blood vessel, such as a vein or artery, of the subject, such as for example the aorta, inferior vena cava, carotid artery, carotid vein, coronary artery, brachial artery, brachial vein, radial artery, radial vein, femoral artery, femoral vein, popliteal artery, popliteal vein, anterior tibial artery, posterior tibial artery, tibioperoneal trunk, peroneal artery, dorsalis pedis artery, renal artery, or renal vein.

Administering the fluorine-18-labeled sodium fluoride to a subject during step (a) can be performed by any suitable means. In some instances, step (a) is performed by administering the fluorine-18-labeled sodium fluoride via intra-venous or intra-arterial injection or infusion. Methods and conditions needed for administering agents, such as fluorine-18-labeled sodium fluoride, in suitable pharmaceutical formulations and at desired dosages are known the person of ordinary skill in the art.

Use of digital PET/CT systems for dynamic PET imaging using a fluorine-18-labeled sodium fluoride can improve spatial and/or temporal image resolution while also reducing radionuclide dosing. The methods described herein can be used for pre-clinical and/or clinical applications.

### Examples

### Example 1: Dynamic PET Imaging Using ¹⁸F-NaF for Absolute Quantification of Skeletal Muscle Perfusion

### Materials and Methods

Surgical Induction of Hindlimb Ischemia in Porcine Model Eight male Yorkshire pigs (10.31±0.92 kg) were sedated via intramuscular administration of ketamine (15-20 mg/kg) and xylazine (1-2 mg/kg), and then intubated and mechanically ventilated with 100% oxygen (VentiPAC, Smiths Medical International Ltd., Luton, UK). Propofol was intravenously infused at 10-30 mg/kg/hour through an ear vein catheter for maintenance of anesthesia levels during all procedures. Depth of anesthesia was monitored every 15 minutes by evaluating the animal's blink reflex and jaw tone. Blood pressure, heart rate, oxygen saturation, and body temperature were continuously monitored during all surgical and imaging procedures (IntelliVue MP30; Philips Healthcare, Andover, MA). The body temperature was maintained above 37 degrees Celsius during all surgical and imaging procedures to control for the potential impact of temperature on subsequent perfusion measures. Animals were fasted prior to surgery and imaging for a minimum of 8 hours. To induce hindlimb ischemia, animals underwent surgical cutdown and permanent unilateral ligation of the right femoral artery, as previously described (Stacy M R, et al., Circ Cardiovasc Imaging. 2014; 7:92-99). Prior to PET/CT imaging, jugular vein and carotid artery access were established via percutaneous puncture and placement of 4-F polyethylene catheters (Cook Medical, Bloomington, IN), which enabled venous administration of ¹⁸F-NaF for PET imaging and continuous arterial blood sampling during PET data acquisition. Experimental protocols were approved by the Institutional Animal Care and Use Committee at Nationwide Children's Hospital and were in compliance with the Association for Assessment and Accreditation of Laboratory Animal Care International policies.

### Preclinical PET/CT Image Acquisition and Reconstruction

Dynamic PET imaging was performed under resting conditions on the day of femoral artery occlusion and two weeks after arterial occlusion using a hybrid PET/CT system (Discovery 690, GE Healthcare, Chicago, IL). Co-registered CT images were acquired with a matching slice thickness of 3.27 mm, at 120 kVp and 300 mA, for the purposes of PET attenuation correction and calf muscle segmentation. A 2.5-minute dynamic PET scan was started in list-mode immediately before intravenous administration of ¹⁸F-NaF (182.8±14.4 MBq), with the dose and subsequent saline flush administered over 20 seconds.

The 60-minute dynamic PET scan was started in list-mode immediately before intravenous administration of ¹⁸F-FDG (196.8±7.5 MBq), with the radioisotope dose and subsequent saline flush continually administered over a 20 second time frame. The lower abdomen and both hindlimbs were positioned within the PET camera's field-of-view (FoV) to include both the abdominal aorta and muscles of interest. The dynamic PET data was reconstructed using 2 iterations and 32 subsets of the ordered subset expectation and maximization algorithm with trans-axial reconstruction using full-width-at-half-maximum Gaussian filter set to 4.5 mm. Dynamic PET images were reconstructed at 3-second intervals for the acquisition of an arterial input function (AIF) from the abdominal aorta, and 5-second intervals for analysis of ¹⁸F-NaF uptake kinetics in calf skeletal muscle. PET image volumes were preprocessed using Hounsfield-unit based attenuation correction. The PET image reconstruction matrix was 192×192 pixels, with an in-plane voxel size of 3.65×3.65 mm and slice thickness of 3.27 mm. CT images were acquired with a slice thickness of 3.27 mm, at 120 kVp and 300 mA, for attenuation correction and calf muscle segmentation.

### Validation of Image-Derived Arterial Input Function

Arterial blood was sampled from the carotid artery in 6 animals during dynamic PET acquisition to generate correction factors for an image-derived AIF. Specifically, 1 mL of arterial blood was sampled every 5 seconds for the first 90 seconds and every 10 seconds for the following 60 seconds of PET imaging at a constant rate (12 mL/min) using a Harvard peristaltic pump (Harvard Apparatus, South Natick, MA). Approximately 0.5 mL of each sample was used to measure whole blood radioactivity while the other 0.5 mL of blood was centrifuged at 2000 g for 15 minutes to acquire plasma radioactivity. Whole blood and plasma samples were weighed, counted, and corrected for radioactive decay using a gamma well counter (WIZARD², PerkinElmer, Waltham, MA). Whole blood and plasma time activity curves (TACs) were then obtained, as well as plasma—to whole blood ratios for each arterial sample.

Three PET phantom imaging studies were performed for cross-calibration of count detection between the PET camera and gamma counter. Specifically, a 4-liter, 20-cm cylindrical phantom was filled with a uniform solution composed of various concentrations of ¹⁸F (0.81 MBq/L, 1.41 MBq/L, and 1.86 MBq/L). PET phantom imaging was then performed using the same image acquisition and reconstruction parameters used for in vivo imaging studies. Three volumes of interest (VOIs) (also referred to as regions of interest (ROIs)) of equal size were manually drawn within the phantom, and the average concentrations of ¹⁸F were calculated for each VOL. Values for the three VOIs were then averaged to represent the overall ¹⁸F concentration for the PET phantom. After imaging, an aliquot of solution (1.5 mL) for each ¹⁸F concentration was sampled from the phantom and used for gamma counting to cross-calibrate the counts quantified by PET image analysis and gamma counting. In addition to arterial blood sampling for gamma counting, plasma samples were analyzed for glucose concentration at baseline (prior to initiation of PET imaging) and every 10 minutes during image acquisition (i.e., 10-, 20-, 30-, 40-, 50-, and 60-minute time point). All measurements of plasma glucose concentration were assessed using a commercially available blood glucose monitor (AlphaTRAK 2; Zoetis Inc., NJ, USA).

To address potential limitations of partial volume effect and spill-over effects for the image-derived AIF, VOIs were defined for both the artery (providing the arterial TAC function IMG(t)) and the adjacent background (providing the background TAC function B(t)). Using manually sampled arterial blood, the true arterial TAC (i.e., C(t)) was derived by fitting the data to Equation 1 below:

$$IMG ( t ) = α \times C ( t ) + β \times B ( t ) \qquad ( Eq . 1 )$$

where α is the partial volume correction (PVC) factor and R is the background activity in the arterial VOI (spill-over correction factor). The VOI used to generate the IMG(t) was defined using an early dynamic frame in which the bolus of activity was best visualized. A VOI was placed over the pixel with maximum intensity and a TAC generated by averaging over the whole volume of the abdominal aorta VOL. The PVC factor was estimated from an annular background VOI surrounding the aorta and calculated using arterial blood sampling. Least-squares fitting was then used to determine a value for α and β to minimize the difference between the left- and right-hand sides of Equation 1. After determination of α and β, the AIF was inferred by substituting the TACs for IMG(t) and B(t) in Equation 1 to solve for C(t) (Puri T, et al., Nucl Med Commun. 2011; 32:808-817).

### Preclinical Dynamic PET/CT Image Processing

PET data kinetic modeling was performed using whole blood data to acquire an image-derived AIF from the abdominal aorta. A whole-blood time activity curve (TAC) was generated by drawing a 100 mm³ VOI in the abdominal aorta. Imaged-derived whole blood TAC was then corrected to true arterial TAC C(t) using Equation 1 and converted to plasma TAC using Equation 1 (shown above), where α was the partial volume correction (PVC) factor and β was the background activity in the arterial volume of interest (VOI) (spill-over correction factor). A deterministic multi-compartment model with 3 exponential components was considered for fitting the plasma TAC to generate an AIF (PMOD Technologies LLC, version 4.1, Zurich, Switzerland). Rigid co-registration transformations were applied to register PET and CT images, and calf muscle VOIs were drawn on co-registered CT attenuation images, as previously described (Stacy M R, et al., Circ Cardiovasc Imaging. 2014; 7:92-99). For evaluation of skeletal muscle perfusion, the first 2.5 minutes of the PET list data was reconstructed in 3-second frames for the acquisition of an image-derived arterial input function (AIF) from the abdominal aorta, and 5-second frames for analysis of ¹⁸F-FDG uptake kinetics in the bilateral calves. Absolute measures of skeletal muscle perfusion were computed via one-tissue compartment kinetic modeling using the plasma AIF and skeletal muscle TACs, which assumed the venous outflow of ¹⁸F-NaF from skeletal muscle was zero and the first-pass extraction of ¹⁸F-NaF in skeletal muscle was 100%. To control for potential differences in hemodynamic loading conditions across animals, perfusion values were normalized by rate pressure product (RPP) as follows: the perfusion value was multiplied by the reference RPP for the entire animal cohort and then divided by the individual RPP at the time of imaging.

For evaluation of skeletal muscle metabolism, the entire 60-minute PET list data was reconstructed using the following sequence: 6 frames of 20 seconds each, followed by 3 frames of 60 seconds each, 3 frames of 300 seconds each, and 4 frames of 600 seconds each, as previously described [Chou T H, Nabavinia M, Tram N K, Rimmerman E T, Patel S, Musini K N, et al., Quantification of skeletal muscle perfusion in peripheral artery disease using 18F-sodium fluoride positron emission tomography imaging. J Am Heart Assoc. 2024]. The overall rate of ¹⁸F-FDG uptake (Ki) of the ischemic and control calf was quantified from PET imaging with a 3-tissue compartment model developed by Bertoldo et al. [Bertoldo A, Peltoniemi P, Oikonen V, Knuuti J, Nuutila P, Cobelli C. Kinetic modeling of [(18)F]FDG in skeletal muscle by PET: a four-compartment five-rate-constant model. Am J Physiol Endocrinol Metab. 2001; 281:E524-36] using an image derived AIF and calf muscle TACs. The metabolic rate of glucose (MRGlu) in the calves was then calculated by multiplying the image-derived Ki by the plasma glucose concentration and correcting for the relative metabolism of ¹⁸F-FDG versus metabolism of true glucose using the lumped constant, which was assumed to be 1.2 for skeletal muscle based on findings of Kelley, et al. [Kelley D E, Williams K V, Price J C, Goodpaster B. Determination of the lumped constant for [18F]fluorodeoxyglucose in human skeletal muscle. J Nucl Med. 1999; 40:1798-804]. The MRGlu for was ultimately expressed as μmol/min/kg of tissue.

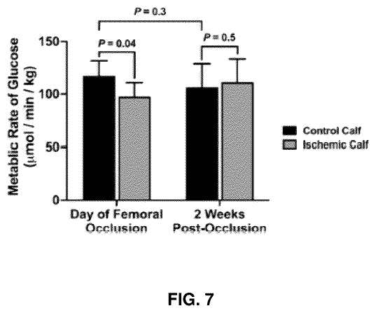

The overall rate of ¹⁸F-FDG uptake (Ki) was significantly reduced in the ischemic calf compared to the control calf on the day of femoral artery occlusion (0.014±0.005 ml/min/g versus 0.017±0.005; P=0.004) and recovered to control levels 2 weeks after femoral artery occlusion (ischemic calf, 0.016±0.008 ml/min/g versus control calf, 0.016±0.007 ml/min/g; P=0.5). Plasma glucose concentrations did not differ for the first and second PET/CT imaging sessions (first scan, 8.3±1.2 mmol/L versus second scan, 8.0±0 0.8 mmol/L; P=0.5). Skeletal muscle MRGlu was significantly reduced in the ischemic calf compared to the control calf on the day of occlusion (97.6±35.3 μmol/min/kg versus 117.2±38.3 μmol/min/kg; P=0.001) (FIG. 7). Measures of skeletal muscle MRGlu recovered to control levels two weeks after femoral artery occlusion (ischemic calf, 111.3±58.6 μmol/min/kg vs. control calf, 106.7±58.5 μmol/min/kg; P=0.2) (FIG. 7).

### Quantification of Skeletal Muscle Microvascular Remodeling in Porcine Model

Following completion of PET/CT imaging at 2 weeks post-occlusion, animals were euthanized via intravenous administration of euthasol (1 ml/4.5 kg), and muscle samples (˜20 grams) were immediately harvested from the gastrocnemius muscle of the bilateral calves in 7 of 8 pigs to evaluate the potential connection between calf muscle angiogenesis and 2-week perfusion recovery. Anatomical sampling site was standardized for each muscle sample for the assessment of capillary density. Samples were fixed with 4% paraformaldehyde and paraffin embedded, and then sectioned at 4-μm thickness. Sections were stained with primary Anti-CD31 (ab28364, Abcam, Cambridge, U.K.) and anti-smooth muscle actin (α-SMA) (Agilent M0851, Santa Clara, CA). Secondary antibodies used were Alexa Fluor 488 IgG1 goat anti-mouse (A32723, Thermo Fisher Scientific, Waltham, MA) and Alexa Fluor 580 IgG goat anti-rabbit (A-11011, Thermo Fisher Scientific). Sections were washed 3 times in PBS and slides were cover slipped using ProLong Gold Antifade Mountant with DAPI (Thermo Fisher Scientific). Suitable cross-sections for analysis contained nearly circular fibers and clear round-shaped capillaries. Image quantification was performed using ImageJ (National Institutes of Health) by blinded observers in images acquired at ×20 magnification using a minimum of six images per muscle (BZ×800 fluorescent microscope, Keyence, Itasca, IL). Capillary density was expressed as capillary-to-muscle fiber ratios and arteriole density was expressed as the percent of positive α-SMA stained muscle area.

### Validation of PET Imaging Methodology in Porcine Model

To validate dynamic PET imaging methodology, microsphere-derived measurements of limb perfusion were acquired during four acute PET/CT imaging studies in Yorkshire pigs (9.88±0.25 kg). Animals first underwent unilateral femoral artery occlusion and jugular vein and carotid artery catheterization. Animals then underwent left thoracotomy and catheterization of the left atrium using a 6-French balloon wedge pressure catheter (Teleflex, Morrisville, NC). Specifically, a small incision was made in the left atrium, the catheter was inserted directly into the atrium and secured with 5-0 prolene sutures, and the chest wall was sutured closed prior to imaging. At the start of PET data acquisition, approximately 6.25×106 (2.5 mL) stable-labeled microspheres (15 μm gold microspheres; BioPAL, Inc.; Worcester, MA) were injected via the left atrial catheter in conjunction with administration of ¹⁸F-NaF (200.5±5.6 MBq) for dynamic PET imaging of muscle perfusion. The microsphere quantity and preparation followed vendor's instructions and previously published large animal microsphere studies of hindlimb skeletal muscle (Fischman A J, et al., J Appl Physiol. 2002; 92:1709-1716). Fifteen seconds before microsphere administration and PET imaging, a one cubic centimeter (cc) reference arterial blood sample was collected. Arterial blood was then continuously sampled throughout the 2.5-minute PET data acquisition using a Harvard peristaltic pump (Harvard Apparatus, South Natick, MA) at a constant rate (3 mL/min).

Following all PET imaging and microsphere procedures, animals were euthanized via intravenous administration of euthasol (1 mL/4.5 kg), and tissue samples (˜20 grams) were immediately collected from multiple muscle groups of the ischemic and control hindlimbs (i.e., gracilis, semimembranosus, gastrocnemius, and soleus) to evaluate agreement level between PET- and microsphere-derived measures of resting perfusion for each muscle group. Muscle samples were weighed, underwent full radioactive decay for ¹⁸F, and were then shipped to the microsphere vendor for processing of microsphere concentrations and perfusion calculations for each muscle, which were expressed as mL/min/g. Skeletal muscle perfusion was calculated using the reference sample technique, assuming 100% first-pass extraction of microspheres, as described in Fischman A J, et al., J Appl Physiol. 2002; 92:1709-1716.

### Clinical Dynamic PET/CT Imaging

Patients with PAD (n=39) were prospectively enrolled for dynamic ¹⁸F-NaF PET/CT imaging as part of an ongoing study evaluating the utility of nuclear perfusion imaging techniques in PAD patients (NCT03622359). Patients were at least 18 years of age, with a history of significant obstructive disease for one or multiple lower extremity arteries, as identified by prior abnormal ABI, TBI, CT angiography, or Duplex ultrasound. The study protocol was approved by the Nationwide Children's Hospital Institutional Review Board for Human Subjects Research and the Radiation Safety Committee. The protocol was in accordance with the guidelines set forth by the Declaration of Helsinki.

Dynamic PET/CT images were acquired in patients following a 4-hour fasting period that also consisted of abstinence from caffeine and alcohol. Images of the lower extremities were acquired under resting conditions using the same clinical PET/CT system and protocol as in porcine studies. Specifically, both calves of patients were positioned within the PET camera FoV and a 2.5-minute dynamic PET scan was initiated in list-mode immediately before ¹⁸F-NaF (341.51±40.33 MBq) was intravenously administered over 20 seconds. The proximal end of the PET FoV was set at the top of tibia for all subjects for standardization of anatomical scan coverage. Co-registered CT images were acquired with a slice thickness of 3.27 mm, at 120 kVp, and 20 mA for attenuation correction of PET data and segmentation of the gastrocnemius, soleus, and tibialis anterior muscles of the calf. The dynamic PET data was reconstructed using the same methods as described above for porcine studies.

A whole blood TAC was generated by drawing a 100 mm³ VOI in the popliteal artery of each leg using the frame with highest PET intensity, and an annular background VOI surrounding the popliteal artery was drawn to create a background TAC. Given that the diameter of the human popliteal artery was similar to the abdominal aorta of the porcine model, the VOI sizes and surrounding background measures used for humans and pigs were identical. Therefore, the partial volume and spill-over correction factors generated from the porcine study were applied to correct image-derived TACs for clinical studies. After partial volume and spill-over correction, whole blood TACs were converted to plasma TACs using a 1.3 plasma-to-whole blood ratio, as previously reported for humans (Schiepers C, et al., J Nucl Med. 1997; 38:1970-1976). Next, a deterministic multi-compartment (triple-exponential) model was assumed for fitting the plasma TAC to generate the plasma AIF. Once modeled, plasma AIF of right and left legs were regressed against the raw whole blood activity curves, and the Chi-square was computed between the modeled AIF and raw data to determine the goodness of fit for each leg. The AIF with lower Chi-square was used for both legs, which allowed for identifying the most accurate whole blood inputs. VOIs for the gastrocnemius, soleus, and tibialis anterior muscle groups were segmented using CT images, which allowed for muscle-specific TACs for kinetic modeling and perfusion calculation, as described for porcine studies.

### Pixelwise Mapping of Skeletal Muscle Perfusion

Pixelwise perfusion maps (i.e., K1 constant maps) were generated for porcine and clinical studies by parametric mapping (PMOD Technologies LLC, Zurich, Switzerland). A linear analytical solution was assumed, as in previous magnetic resonance arterial spin labeling imaging studies evaluating muscle perfusion (Pollak A W, et al., JACC Cardiovasc Imaging. 2012; 5:1224-1230). It was assumed that the same linear solutions could be adopted for dynamic ¹⁸F-NaF PET imaging to yield volumetric perfusion maps of skeletal muscle.

### Lower Extremity Hemodynamic Measures and Disease Classification

Ankle- and toe-brachial indices (ABI, TBI) as well as great toe pressures were measured in both lower extremities of patients following 20 minutes of rest in the supine position (Vicorder, Skidmore Medical Ltd., Bristol, UK). All measurements were performed by the same experienced investigator to limit variability between subjects. In addition to hemodynamic measures, each patient was assigned a Rutherford Classification (Rutherford R B, et al., J Vasc Surg. 1997; 26:517-538) ranging from stage 0 to 6 based on their self-reported and chart-documented clinical symptoms of PAD at the time of PET imaging. Patients were then grouped into one of three categories based on their Rutherford Classification, which consisted of patients experiencing stage 0-1 (asymptomatic to mild claudication symptoms), stage 2-3 (moderate to severe claudication), and stage 4-6 (rest pain to severe ischemic ulcers) symptoms.

### Statistical Analysis

Serial changes in hindlimb perfusion were evaluated using a two-way repeated measures analysis of variance (ANOVA). Differences in capillary and arteriole density between control and ischemic hindlimb muscles were evaluated using paired t-tests. Agreement between PET perfusion and gold-standard microsphere measures were assessed using Bland-Altman plot and concordance correlation coefficient (CCC). Differences in calf perfusion for each Rutherford classification category were compared using one-way ANOVA, with Tukey's method used to account for multiple comparisons. To assess the relationship between PET perfusion and traditional (i.e., ABI, TBI, toe pressure) measures and disease severity (i.e., Rutherford Classification), ordered logistic regression analyses were performed. A p value of <0.05 was considered statistically significant for all analyses. Estimates are presented with 95% confidence intervals (CI). Statistical analyses were performed using R, version 4.2.0 (R Core Team, 2022) or Prism for macOS, version 9.3.0 (GraphPad Software, LLC).

### Results

### Validation of Image-Derived Arterial Input Function

Phantom imaging studies demonstrated a linear relationship between count detection from PET imaging and gamma counting, with the resulting equation for line of best fit being y=0.5905x+0.1363, where x represented counts detected by gamma counting and y represented PET-derived counts. For abdominal aorta TACs derived from porcine studies, the best fitting value was an a value of 0.86 for the partial volume correction factor and a β value of 0 for the spill-over correction factor. The plasma-to-whole blood ratio did not change in blood samples continuously acquired during the 2.5-minute dynamic PET scan. Therefore, the mean plasma-to-whole blood ratio (1.13±0.05) calculated from 6 porcine studies was used to correct whole-blood PET data for pre-clinical imaging studies.

### Serial In Vivo Dynamic ¹⁸F-NaF PET/CT Perfusion Imaging

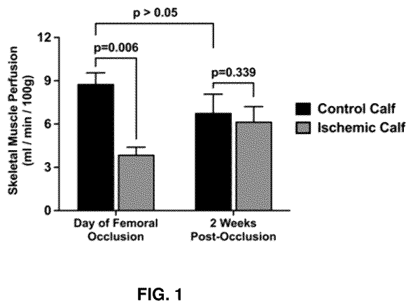

Dynamic ¹⁸F-NaF PET/CT imaging quantified significant reductions in calf skeletal muscle perfusion following unilateral femoral artery occlusion (ischemic calf, 3.88±1.46 mL/min/100 g versus control calf, 8.78±2.17 mL/min/100 g; P=0.001) (PET imaging not shown). Measures of calf skeletal muscle perfusion recovered to control levels two weeks after femoral artery occlusion (ischemic calf, 6.18±2.93 mL/100 g/min vs. control calf, 6.78±3.66 mL/100 g/min; P=0.339) (see FIG. 1).

### Analysis of Skeletal Muscle Microvascular Remodeling

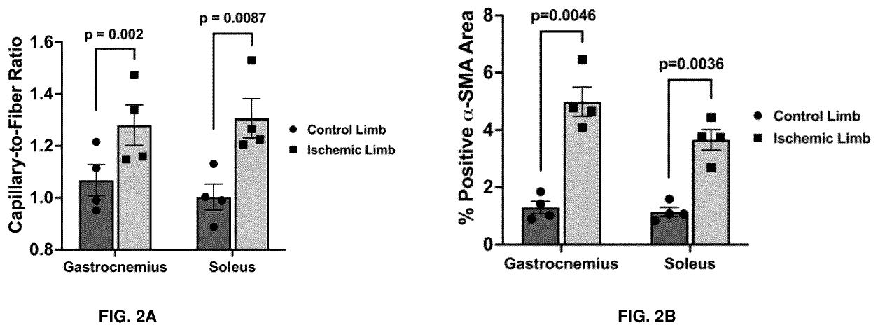

Immunofluorescence analyses demonstrated that relative capillary density (expressed as capillary-to-muscle fiber ratio) was significantly increased two weeks after induction of unilateral hindlimb ischemia for both the gastrocnemius (ischemic limb ratio, 1.28±0.16 vs. control limb ratio, 1.07±0.12; P=0.002) and soleus (ischemic limb ratio, 1.31±0.15 vs. control limb ratio, 1.0±0.10; P=0.008) muscles of the calf (see FIG. 2A). Additionally, arteriole density (expressed as percent positive α-SMA stained area) was significantly increased two weeks after induction of ischemia for both the gastrocnemius (ischemic limb: 4.99±1.02% vs. control limb: 1.3±0.43%; P=0.0046) and soleus (ischemic limb: 3.66±0.72% vs. control limb: 1.14±0.31%; P=0.0036) muscles (see FIG. 2B).

### Validation of Dynamic 18F-NaF PET Imaging Method

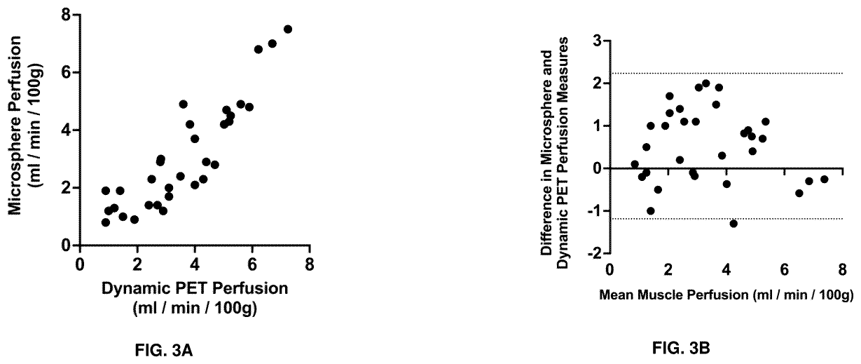

Microsphere studies demonstrated strong agreement between absolute measures of muscle perfusion calculated from microsphere and dynamic ¹⁸F-NaF PET imaging, which was confirmed by a concordance correlation coefficient (CCC) of 0.85 (95% CI: 0.76, 0.90) between measures obtained by both techniques (see FIG. 3A). Bland-Altman analysis revealed a mean difference of 0.52 with 95% agreement limits of −1.19 to 2.2 for perfusion measures acquired from microsphere and dynamic PET methods (see FIG. 3B).

### Clinical Subject Characteristics

Patients with PAD and CLTI presented with numerous cardiovascular comorbidities and risk factors. Detailed characteristics for each patient group are summarized in Table 1 below. One research subject in the CLTI patient group included a 44-year-old male patient with DM and CLTI who underwent dynamic ¹⁸F-NaF PET/CT perfusion imaging before and 6 days after endovascular therapy of the left superficial femoral artery using a 5 mm×12 mm balloon and 6 mm×140 cm self-expanding drug-coated stent.

TABLE 1
Patient Characteristics

| | | |
|---|---|---|
| Rutherford | Rutherford | Rutherford |
| 0-1 | 2-3 | 4-6 |
| N = 15 | N = 12 | N = 12 |

Demographics

| Age (years) | 65.0 ± 11.1 | | 61.5 ± 10.1 | | 57.7 ± 10.9 | |
|---|---|---|---|---|---|---|
| Sex (Male) | 5 | (33.3%) | 7 | (58.3%) | 8 | (66.7%) |
| Race | | | | | | |
| White | 8 | (53.3%) | 8 | (66.7%) | 5 | (41.7%) |
| Black | 7 | (46.7%) | 3 | (25.0%) | 7 | (58.3%) |
| Refused to answer | 0 | (0%) | 1 | (8.3%) | 0 | (0%) |
| Ethnicity | | | | | | |
| Hispanic or Latino | 0 | (0%) | 0 | (0%) | 0 | (0%) |
| Not Hispanic or Latino | 15 | (100%) | 12 | (100%) | 15 | (100%) |
| Body mass index | 28.9 ± 7.7 | | 31.4 ± 6.7 | | 30.1 ± 7.6 | |
| (kg/m²) | | | | | | |
| Ankle-Brachial Index | 0.55 ± 0.36 | | 0.60 ± 0.29 | | 0.49 ± 0.53 | |
| Toe-Brachial Index | 0.25 ± 0.24 | | 0.37 ± 0.22 | | 0.25 ± 0.27 | |
| Toe Pressure (mmHg) | 35.9 ± 33.7 | | 51.9 ± 29.2 | | 32.2 ± 34.2 | |
| Systolic Blood | 133.2 ± 20.1 | | 144.9 ± 11.7 | | 140.5 ± 26.4 | |
| Pressure (mmHg) | | | | | | |
| Diastolic Blood | 72.3 ± 10.1 | | 76.3 ± 13.1 | | 77.3 ± 11.9 | |
| Pressure (mmHg) | | | | | | |
| Fasting Glucose | 129.0 ± 45.8 | | 114.8 ± 56.5 | | 135.5 ± 73.4 | |
| (mg/dl) | | | | | | |
| Hemoglobin A1C (%) | 6.4 ± 0.8 | | 6.6 ± 1.1 | | 7.5 ± 2.1 | |
| Total Cholesterol | 161.8 ± 52.3 | | 154.2 ± 43.5 | | 143.6 ± .5.9 | |
| (mg/dL) | | | | | | |
| Statin Use | 13 | (86.7%) | 9 | (75.0%) | 9 | (75.0%) |

Comorbidities/Risk Factors

| | | | | | | |
|---|---|---|---|---|---|---|
| Diabetes Mellitus | 11 | (73.3%) | 5 | (41.7%) | 10 | (83.3%) |
| Chronic Kidney | 4 | (26.7%) | 1 | (8.3%) | 6 | (50.0%) |
| Disease | | | | | | |
| Coronary Artery | 7 | (46.7%) | 4 | (33.3%) | 3 | (25.0%) |
| Disease | | | | | | |
| Tobacco Use | 6 | (40.0%) | 4 | (33.3%) | 4 | (33.3%) |

Values given in Table 1 are mean ± SD or n (%)

Patients presented with numerous cardiovascular comorbidities and risk factors. Detailed characteristics for each patient subgroup are summarized in Table 1 above. One additional research subject included a 44-year-old male patient with DM and PAD who underwent dynamic ¹⁸F-NaF PET/CT perfusion imaging before and 6 days after endovascular therapy of the left superficial femoral artery using a 5 mm×12 mm balloon and 6 mm×140 cm self-expanding drug-coated stent.

### Clinical Translation of Dynamic 18F-NaF PET Perfusion Imaging

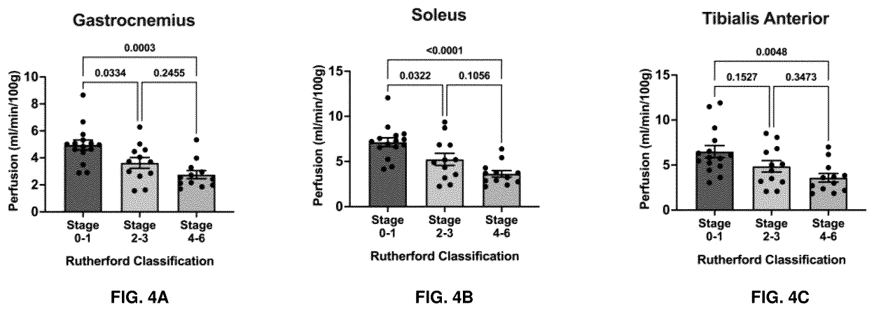

Dynamic ¹⁸F-NaF PET imaging demonstrated qualitative differences in resting calf muscle perfusion between symptomatic and asymptomatic limbs in patients with PAD (PET imaging not shown). Fused ¹⁸F-NaF PET/CT perfusion images also allowed for noninvasive detection of abnormalities in calf muscle perfusion that were anatomically matched with regions of muscle atrophy (PET imaging not shown). Grouping of patients based on claudication symptoms and disease stage (i.e., Rutherford Classification) revealed that PET-derived skeletal muscle perfusion values for each calf muscle group were significantly decreased with increasing severity of PAD (see FIGS. 4A-4C).

Ordered logistic regression analyses revealed that PET-derived measures of skeletal muscle perfusion were significantly associated with Rutherford Classification for the gastrocnemius (p=0.0006), soleus (p=0.0004), and tibialis anterior (p=0.017) calf muscles (Table 2 below). Specifically, a 1 unit decrease in gastrocnemius perfusion (i.e., 1 ml/min/g) increased the odds of having a higher Rutherford Classification score by 55.61%, a 1 unit decrease in soleus perfusion increased the odds of having a higher Rutherford score by 42.67%, and a 1 unit decrease in tibialis anterior perfusion increased the odds of having a higher Rutherford score by 25.01%. Traditional hemodynamic measures for PAD, including ABI (p=0.78), TBI (p=0.79), and toe pressure (p=0.96), were not significantly associated with Rutherford Classification (Table 2 below).

TABLE 2
Association Between PET Perfusion Values, Traditional
PAD Measures, and Patient Rutherford Classification

| | Odds Ratio [95% CI] | P-Value |
|---|---|---|
| Gastrocnemius Perfusion | 0.44 [0.27-0.69] | 0.0006 |
| Soleus Perfusion | 0.57 [0.41-0.77] | 0.0004 |
| Tibialis Anterior Perfusion | 0.75 [0.58-0.94] | 0.017 |
| Ankle-Brachial Index | 0.78 [0.14-4.4] | 0.78 |
| Toe-Brachial Index | 1.38 [0.13-15.39] | 0.79 |
| Toe Pressure | 1.00 [0.98-1.02] | 0.96 |

Odds ratios are based on ordered logistic regression models. CI = confidence interval.

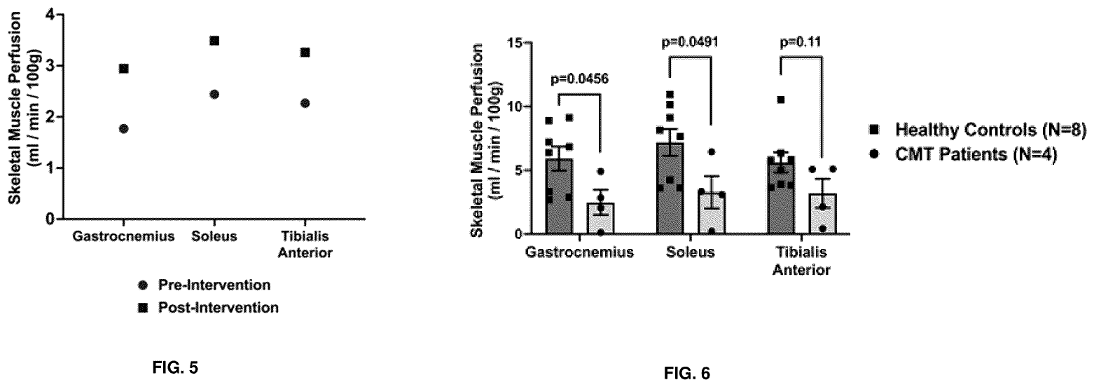

¹⁸F-NaF PET/CT perfusion imaging performed before and after lower extremity endovascular therapy in a patient demonstrated both qualitative (PET imaging not shown) and quantitative (see FIG. 5) increases in skeletal muscle perfusion for the gastrocnemius (before: 1.77 mL/min/100 g; after: 2.94 mL/min/100 g), soleus (before: 2.44 mL/min/100 g; after: 3.49 mL/min/100 g), and tibialis anterior (before: 2.27 mL/min/100 g; after: 3.26 mL/min/100 g).

### Discussion

This example sought to pre-clinically test and clinically translate a dynamic PET imaging framework using ¹⁸F-NaF that would enable absolute quantification of skeletal muscle perfusion in the setting of PAD and create opportunities for the for dual assessment of both perfusion and active vascular microcalcification in patients with PAD. Considering that bone uptake of ¹⁸F-NaF is driven by blood supply, (Czernin J, et al., J Nucl Med. 2010; 51:1826-1829) it was hypothesized that the 1) first-pass kinetics of ¹⁸F-NaF in skeletal muscle would also be dictated by blood supply to the lower extremities and 2) during first pass of ¹⁸F-NaF through the circulation there was a brief time when 100% of ¹⁸F-NaF resides within the muscle capillary bed and the venous outflow of ¹⁸F-NaF is zero, thereby allowing for calculation of skeletal muscle perfusion using a one-tissue compartment model.

Thus, this example demonstrated preclinical testing and translation of a PET imaging approach that quantified absolute measures of skeletal muscle perfusion using a commercially available ¹⁸F-labeled radionuclide.

Dynamic ¹⁸F-NaF PET imaging allowed for quantification of significant reductions in calf perfusion in the setting of limb ischemia and quantified serial improvements in calf muscle perfusion that coincided with increases in muscle microvascular density, thus demonstrating that this method detects abnormalities as well as improvements in microvascular perfusion under resting conditions. Furthermore, dynamic ¹⁸F-NaF PET measures of muscle perfusion agreed with gold-standard microsphere-based measurements of perfusion acquired in multiple muscle groups. The clinical translation of the same dynamic ¹⁸F-NaF PET imaging method further revealed the utility for quantifying regional abnormalities in resting calf muscle perfusion in patients with PAD, and that PET-derived measures of calf muscle perfusion were significantly associated with severity of symptoms in PAD patients. These findings collectively suggest that dynamic ¹⁸F-NaF PET offers an efficient nuclear imaging approach for assessing perfusion, may reduce PET imaging costs by eliminating the need for an on-site cyclotron or rubidium generator, and could increase access to muscle perfusion imaging methods for healthcare systems who possess PET cameras. In addition to these advances, given that dynamic ¹⁸F-NaF PET imaging provides quantitative perfusion within 2.5 minutes, this method provides opportunities for coupling of an initial dynamic PET perfusion scan with a delayed static PET scan for dual assessment of muscle perfusion and active arterial microcalcification in PAD patients using a single dose administration of ¹⁸F-NaF during a single imaging session.

Previous studies have demonstrated that dynamic PET imaging can quantify absolute measures of lower extremity muscle perfusion; however, many of these studies were performed decades ago using on-site cyclotron production of ¹⁵O-water (Burchert W, et al., J Nucl Med. 1997; 38:93-98; Schmidt M A, et al., J Nucl Med. 2003; 44:915-919; Scremin O U, et al., Am J Phys Med Rehabil. 2010; 89:473-486; Fischman A J, et al., J Appl Physiol. 2002; 92:1709-171). ¹⁵O-water has been the most commonly used PET radionuclide for quantifying absolute measures of muscle perfusion in large animal models16 or patients with limb ischemia (Burchert W, et al., J Nucl Med. 1997; 38:93-98; Schmidt M A, et al., J Nucl Med. 2003; 44:915-919; Scremin O U, et al., Am J Phys Med Rehabil. 2010; 89:473-486). Prior ¹⁵O-water studies reported resting perfusion values of the calves and thighs ranging from 2-4 mL/min/100 g ((Burchert W, et al., J Nucl Med. 1997; 38:93-98; Scremin O U, et al., Am J Phys Med Rehabil. 2010; 89:473-486; Fischman A J, et al., J Appl Physiol. 2002; 92:1709-171). While the pre-clinical perfusion values found here were higher than those previously reported using ¹⁵O-water, young healthy animals that were in an active growth phase were used for this study, as opposed to adult humans or canines, which might explain these higher values.

Additionally, unlike prior ¹⁵O-water studies in animals and patients, the present study quantified resting perfusion within specific muscle groups of the legs. Previously shown significant heterogeneity in tissue perfusion and oxygenation between muscle groups of the calves (Chou T H, et al., J Nucl Cardiol. 2020; 27(6):1923-1933; Mahmud S Z, et al., Sci Rep. 2020; 10:6342) were once again found in patients in the present study. This regional heterogeneity in calf perfusion could further explain the diverse and higher range of calf perfusion values quantified in the porcine studies. Recent studies using magnetic resonance imaging (Veit-Haibach P, et al., 2021; 31:5507-5513) and dynamic contrast-enhanced CT (Piert M, et al., J Nucl Med. 2001; 42:1091-1100) methods have reported similar resting perfusion values in the lower extremities of PAD patients, as found with the preclinical ¹⁸F-NaF PET discussed herein, suggesting that perfusion measures acquired by dynamic ¹⁸F-NaF PET may closely align with other emerging imaging techniques.

Arterial cannulation and blood sampling is associated with increased risks for bleeding and injury. Therefore, Example 1 validated an image-derived AIF for dynamic ¹⁸F-NaF PET imaging of muscle perfusion to make dynamic PET imaging more translatable for clinical studies. The plasma-to-whole blood ratio did not change significantly during the course of 2.5 minutes of dynamic PET, and the mean plasma-to-whole blood ratio was 1.13 for the porcine model, which closely agreed with previously reported ratios in porcine studies that were focused on evaluating dynamic ¹⁸F-fluoride PET imaging of bone metabolism.24

The present study demonstrates that dynamic ¹⁸F-NaF PET can quantify absolute measures of limb perfusion, and that these PET-derived measures significantly differ in specific calf muscles with increasing stages of disease. Additionally, it was found that PET-derived measures of calf muscle perfusion were significantly associated with the severity of PAD symptoms, whereas more traditional hemodynamic measures for assessing PAD (i.e., ABI, TBI, and toe pressure) were not. Collectively, these clinical findings suggest that dynamic PET is a sensitive approach for assessing abnormalities in resting perfusion within specific muscle groups that are linked to disease severity.

It was found that absolute measures of perfusion derived from PET imaging were similar between ischemic calf muscles of the porcine model and ischemic calf muscles of PAD patients, indicating that dynamic ¹⁸F-NaF PET detects and quantifies muscle ischemia similarly in animals and humans. This finding further supports the use of dynamic ¹⁸F-NaF PET perfusion imaging for assessing patients with limb ischemia. The use of ¹⁸F-NaF or other standard ¹⁸F-labeled radionuclides for quantitative imaging of muscle perfusion may complement traditional perfusion radionuclides that possess shorter half-lives, thereby eliminating the need for onsite cyclotron production and increasing healthcare access to quantitative PET perfusion imaging for a variety of vascular and musculoskeletal diseases. Additionally, partnering of early-phase dynamic ¹⁸F-NaF (or other standard ¹⁸F-labeled radionuclides) perfusion imaging with delayed static vascular microcalcification imaging allows for molecular imaging in PAD.

### Example 2: Dynamic PET Imaging Using ¹⁸F-FDGfor Absolute Quantification of Skeletal Muscle Perfusion in Patients with Charcot-Marie-Tooth (CMT) Disease

The dynamic PET/CT imaging framework using ¹⁸F-NaF described in Example 1, which allows for absolute quantification of skeletal muscle perfusion, was used in Example 2 with ¹⁸F-FDG to perform regional assessment of calf muscle perfusion in subjects suffering from Charcot-Marie-Tooth (CMT) disease.

Healthy control subjects (n=8) and patients with Charcot-Marie-Tooth (CMT) (n=4) were prospectively enrolled for dynamic ¹⁸F-FDG PET/CT imaging. All subjects were at least 18 years of age. The study protocol was approved by the Nationwide Children's Hospital Institutional Review Board for Human Subjects Research and the Radiation Safety Committee. The protocol was in accordance with the guidelines set forth by the Declaration of Helsinki.

Dynamic PET/CT images were acquired in subjects following a 4-hour fasting period that also consisted of abstinence from caffeine and alcohol. Images of the lower extremities were acquired under resting conditions using the same clinical PET/CT system and protocol previously outlined in porcine and clinical studies using ¹⁸F-NaF. Specifically, both calves of patients were positioned within the PET camera FoV and a 2.5-minute dynamic PET scan was initiated in list-mode immediately before ¹⁸F-FDG was intravenously administered over 20 seconds. The proximal end of the PET FoV was set at the top of tibia for all subjects for standardization of anatomical scan coverage. Co-registered CT images were acquired with a slice thickness of 3.27 mm, at 120 kVp, and 20 mA for attenuation correction of PET data and segmentation of the gastrocnemius, soleus, and tibialis anterior muscles of the calf. The dynamic PET data was reconstructed using the same methods as described above for porcine and clinical studies that utilized ¹⁸F-NaF.

A whole blood TAC was generated by drawing a 100 mm³ VOI in the popliteal artery of each leg using the frame with highest PET intensity, and an annular background VOI surrounding the popliteal artery was drawn to create a background TAC. Given that the diameter of the human popliteal artery was similar to the abdominal aorta of the porcine model, the VOI sizes and surrounding background measures used for humans and pigs were identical. Therefore, the partial volume and spill-over correction factors generated from the porcine study were applied to correct image-derived TACs for clinical studies. After partial volume and spill-over correction, whole blood TACs were converted to plasma TACs using a 1.3 plasma-to-whole blood ratio, as previously reported for humans. Next, a deterministic multi-compartment (triple-exponential) model was assumed for fitting the plasma TAC to generate the plasma AIF. Once modeled, plasma AIF of right and left legs were regressed against the raw whole blood activity curves, and the Chi-square was computed between the modeled AIF and raw data to determine the goodness of fit for each leg. The AIF with lower Chi-square was used for both legs, which allowed for identifying the most accurate whole blood inputs. VOIs for the gastrocnemius, soleus, and tibialis anterior muscle groups were segmented using CT images, which allowed for muscle-specific TACs for kinetic modeling and perfusion calculation, as previously described for porcine and clinical studies using ¹⁸F-NaF.

¹⁸F-FDG PET/CT perfusion imaging in healthy control subjects and patients with Charcot-Marie-Tooth (CMT) revealed qualitatively lower muscle perfusion in the CMT patient (PET imaging not shown).

As shown in FIG. 6, quantitative PET/CT image analysis of perfusion within specific muscles of the calf (gastrocnemius, soleus, and tibialis anterior) demonstrated significantly reduced skeletal muscle perfusion for these multiple muscle groups in the lower extremities of patients with CMT, when compared to healthy control subjects.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific instances of the invention described herein. Such equivalents are intended to be encompassed by the following claims.

We claim:

1. A method for quantifying skeletal muscle perfusion in a tissue of a subject, comprising:
(i) administering to the subject an effective amount of a fluorine-18-labeled radionuclide agent;
(ii) dynamically imaging the subject on a positron emission tomography (PET) system, wherein the imaging begins at least about 1 second prior to step (i); and
(iii) performing kinetic modeling of the PET imaging data following steps (i) and (ii).

2. The method of claim 1, wherein step (ii) is performed for a period of time ranging from about 2 to about 10 minutes.

3. The method of claim 1, wherein step (ii) begins at least about 1 to about 5 seconds prior to step (i).

4. The method of claim 1, wherein the fluorine-18 labeled radionuclide is ¹⁸F-sodium fluoride (NaF) and/or ¹⁸F-2-deoxy-2-fluoro-D-glucose (FDG).

5. The method of claim 1, wherein in step (i), the effective amount of the fluorine-18 labeled radionuclide provides a dose in a range of about 1 mCi to about 10 mCi during at least the first 20 seconds of step (i).

6. The method of claim 1, wherein the tissue is a muscle or group of muscles of the subject.

7. The method of claim 1, wherein step (i) comprises administering the fluorine-18-labeled radionuclide agent via intra-venous or intra-arterial injection or infusion.

8. The method of claim 1, wherein the positron emission tomography (PET) system is a PET/computed tomography (PET/CT) or PET/magnetic resonance (PET/MR) system.

9. The method of claim 1, further comprising, subsequent to step (ii),
(iv) generating at least one pixelwise perfusion map of the tissue, by parametric mapping.

10. The method of claim 1, wherein the patient has a disease selected from the group consisting of a neuromuscular disease, an ischemic disease, and a degenerative muscle disease.

11. The method of claim 10, wherein the ischemic disease is selected from the group consisting of peripheral artery disease (PAD), Charcot-Marie-Tooth syndrome (CMT), exertional compartment syndrome, ischemic myopathy, ischemic rhabdomyolysis, chronic limb-threatening ischemia (CLTI), ischemic necrosis, ischemic fasciitis, and ischemic myositis, or a combination thereof.

12. The method of claim 1, wherein the method is performed on the subject prior to and following a surgical or endovascular revascularization procedure.

13. The method of claim 1, wherein the method is performed on the subject prior to and following a regenerative medicine therapy.

14. The method of claim 1, wherein the kinetic modeling is one-tissue compartment or three-tissue compartment kinetic modeling.

15. A method for quantifying skeletal muscle metabolism in a tissue of a subject, the method comprising the steps of:
(i′) administering to the subject an effective amount of a fluorine-18-labeled glucose or analog thereof;
(ii′) dynamically imaging the patient on a positron emission tomography (PET) system for about 5 to 60 minutes following step (i′); and
(iii′) performing three-tissue compartment kinetic modeling of the PET imaging data following steps (i′) and (ii′).

16. The method of claim 15, wherein the fluorine-18-labeled glucose or analog thereof is selected from the group consisting of ¹⁸F-2-deoxy-2-fluoro-D-glucose (FDG), ¹⁸F-FDGal (fluorodeoxygalactose), ¹⁸F-FDMan (fluorodeoxymannose), ¹⁸F-FET (fluoroethyltyrosine), and ¹⁸F-FDOPA (fluorodihydroxyphenylalanine).

17. The method of claim 15, wherein the fluorine-18-labeled glucose or analog thereof is ¹⁸F-2-deoxy-2-fluoro-D-glucose (FDG).

18. A method for quantifying active vascular calcification in a subject's blood vessel, comprising:
(a) administering to the subject an effective amount of fluorine-18-labeled sodium fluoride;
(b) dynamically imaging the subject using a positron emission tomography (PET) system; and
(c) following step (b), imaging the subject a second time using the positron emission tomography (PET) system, wherein a second dose of fluorine-18-labeled sodium fluoride or another fluorine-18-labeled radionuclide agent is not administered to the subject between step (b) and (c).

19. The method of claim 18, wherein step (b) is performed at least about 10 seconds to about 20 minutes following step (a).

20. The method of claim 18, wherein the imaging in step (c) is performed at least about 45 minutes to about 75 minutes following step (a).

21. The method of claim 18, wherein in step (a), the effective amount administered of the fluorine-18-labeled sodium fluoride provides a dose in a range of about 1 mCi to about 10 mCi.

22. The method of claim 18, wherein step (a) comprises administering the fluorine-18-labeled sodium fluoride via intra-venous or intra-arterial injection or infusion.

23. The method of claim 18, wherein the positron emission tomography (PET) system is a PET/computed tomography (PET/CT) or PET/magnetic resonance (PET/MR) system.
